(19) United States
(12) Patent Application Publication
(10) Pub. No.: US 2023/0358739 A1
(43) Pub. Date: Nov. 9, 2023

(54) IMPROVEMENTS IN OR RELATING TO ASSAY TIMING

(71) Applicant: Vidya Holdings Ltd, London (GB)

(72) Inventors: Stefan Leo Van Workum, London (GB); Vladimir Alexander Turek, London (GB); Marko Dorrestijn, London (GB); David R. Klug, London (GB)

(73) Assignee: Vidya Holdings Ltd, London (GB)

(21) Appl. No.: 17/786,440

(22) PCT Filed: Dec. 18, 2020
(86) PCT No.: PCT/GB2020/053271

§ 371 Date: Jun. 16, 2022

(30) Foreign Application Priority Data
Dec. 19, 2019 (GB) 1918900.0

(51) Int. Cl. G01N 33/543; B01L 3/00

(52) U.S. Cl. CPC G01N 33/54386; B01L 3/5023; B01L 3/502753; B01L 2200/147; B01L 2200/16; B01L 2300/0681; B01L 2300/069; B01L 2300/0816; B01L 2300/123; B01L 2400/0481

(57) ABSTRACT

A sample management module for receiving a fluid sample for an immunoassay is provided. The module comprises: a sample reception device for receiving a sample; a detection reagent; a controller configured to initiate a reaction timer on the basis of an actuation event when the sample and detection reagent are brought into contact.

The present invention relates to improvements in or relating to assay timing and, in particular, to assay timing in a domestic application.

Traditionally, biological assays on relatively large samples, in the range of one to several millilitres, are conducted in strictly controlled environments by skilled operatives such as laboratory technicians or, alternatively, by using an automated computer controlled system, such as an automated pipetting robot. The most common bodily fluid tested is blood because the relevant biomarkers are present at relatively easily detectable levels and the strictly controlled environment and skilled operatives are compatible with the biohazardous aspect of dealing with blood samples.

Moving from a laboratory setting to a domestic application raises a number of problems including that extracting, processing and disposing of blood samples represents a biohazard in a domestic setting. In this context, the term domestic setting or application is intended to encompass any non-clinical environment such as the home, workplace, pharmacy or doctor’s surgery.

There are some blood tests that are performed in the point of care environment. These typically use a small volume of blood such as a finger prick test. However, this procedure is intrusive and painful and therefore can have compliance issues in any circumstance where the test could be deemed non-essential by the user.

Integrated assays, that is assay chips that include all of the reagents and microfluidics to be entirely self-contained, have been developed for use in the domestic environment. These assays obviate the need for the addition of further reagents and also pumping or washing steps, through a careful selection of detection reagents and capture components provided within the assay chip and through the provision of microfluidic configurations that control the flow of a liquid sample provided through the assay chip to combine with detection reagents and capture components under optimum conditions.

However, a key issue around accurate quantitative integrated assays in the domestic or point of care setting relates to the timing of the assay. The signal strength of a diffusion-based assay increase with time as more analyte is captured. Although the assay will eventually reach an equilibrium condition where the assay has effectively been completed, the time period required for this is often incompatible with a point of care application where speed of results is paramount. Therefore, it is important, in this context, to be able to identify positively the time at which the assay commenced and to share this information so that the assay can be managed effectively.

The apparatus commonly used in the laboratory setting are designed to manage high throughput of assay chips and to provide stacking, sorting and a controlled environment in which the assay chips can be incubated. Such apparatus often includes conveyor belts, rotary shelves and transfer station where chips are selected and passed on to a subsequent process or station. These systems often include a crane-like device or a multi-axis robotic arm with grippers or other functional head. These systems need to be operated by a skilled operative and would be entirely out of place in a domestic setting as they would be too large and too complex and their complex operating parameters would likely not be met in the domestic context.

There is therefore a need to provide timing and stacking solutions that allow assays to be accurately timed and fed to a reader in a timely fashion. A stacking solution is required that enables parallel processing of the assay chips because serial processing of assay chips is not compatible with the home, workplace or retail environments that are the targets for this innovation.

It is against this background that the invention has arisen.

According to the present invention there is provided a sample management module for receiving a fluid sample for an immunoassay, the module comprising: a sample reception device for receiving a sample; a detection reagent; a controller configured to initiate a reaction timer on the basis of an actuation event when the sample and detection reagent are brought into contact.

Controlling the timing of an assay is very important and therefore having a reaction timer and a controller to actuate the reaction timer is critical to delivering a device that is capable of bringing immunoassays into the point of care setting. The signal strength of a diffusion-based assay increase with time as more analyte is captured.

Unless there is time for the assay to run to its conclusion, i.e. an equilibrium condition in which no further binding occurs, then the time since the assay commenced is vitally important in calibrated the measured signal. In the point of care setting, obtaining rapid results is key and therefore waiting for the assay to complete is not realistic in this context.

The controller is configured to initiate a reaction timer on the basis of an actuation event which corresponds to when the sample and detection reagent are brought into contact. This may be the lid closure, for example. This positive initiation of the reaction timer contrasts with known systems that provide a sensed commencement of the reaction timer. The provision of one or more sensors to identify the commencement of the reaction and thereby to initiate the reaction timer adds complexity and cost to a single use device. As the geometry of the device is tightly controlled, the flow of the fluid through the device occurs in a repeatable and predictable manner. Therefore the timing of the commencement of the assay can be calculated relative to the time of closure of the lid. The provision of an actuated start point rather than a sensed start point for the assay contributes to the simplification of the device and therefore a reduction in cost of manufacture and simplification of recycling pathways due to a reduction in parts and therefore materials used.

The actuation event may be the closure of the device lid, the operation of a plunger located in the lid of the device, the operation of a pump, the opening of a valve within the device, the opening of a vent within the device, constriction or compression of a wall of the device, or could be the piercing of the lid or wall with a sharp object such as a syringe. The actuation event causes a pressure change or a change in the flow geometry within the sample management module, which provides the dual function of bringing the sample and the detection reagent into contact and actuating the reaction timer. In one embodiment, the opening of a valve or a vent downstream of the sample and detection reagent can be the actuation event. The opening of a valve or vent within the sample management module causes a change in capillary pressure within the sample management module, and brings the sample and detection reagent into contact by enabling a capillary flow.

The reaction timer may or may not actually be included in the sample management module, it simply has to be subject to a controller that is associated with the sample management module. The controller may be active or passive. In some embodiments, where the sample management module takes its simplest form, it may be simply that, included in the readable data is a statement that no local reaction timer is present. Therefore, when the sample management module is interrogated, this information is read and a reaction timer is initiated remote from the sample management module.

The reagent may comprise proteins (antibodies, affibodies, nanobodies), nucleic acids (DNA, RNA), polymers or small molecules any of which could be fluorescently labelled or labelled with nanoparticles.

The detection reagent binds to the target component to form a detection reagent-target component complex. This complex then binds to the capture component to form a sandwich assay. The detection reagent can have inherent light emitting or scattering properties or the detection reagent may have applied to it a label. The detection reagent may be an antibody or an antibody fragment, protein or a peptide, or a nucleic acid.

The label may be one or more of the following: a luminescent entity; a fluorescent entity; a phosphorescent entity; a chemiluminescent entity; an entity that exhibits scattering, such as Rayleigh, Raman or Mie scattering; an entity that exhibits photon upconversion; an enzyme and its substrate that together produce an optical signal such as a luminescent signal and any entity providing a colorimetric signal regardless as to process but specifically exemplified by change to absorption cross section or extinction. In this context, the term upconversion is used to denote any emission following a multi-photon excitation process and this includes two photon fluorescence particles.

In this context, the term entity is used to refer to one or more of the following: a molecule; a cell or cell fragment such as a fragment of cell membrane; an ion; a particle which may be metallic, organic, inorganic or polymeric; a nanoparticle; a cluster, or a quantum dot.

The reagent may be a detection reagent configured to bind with the target components in the sample fluid and move to capture components provided in the vicinity of the detection reagents. All of the reagents required are provided within the sample management module from the outset. This ensures that no further reagents have to be introduced into the module after the sample has been provided.

The sample reception device may include a pad of material, which may be a sponge. The material may be porous such that the sample can move through the pores to exit to pad, whereas particulate contaminants will be retained in the pad. The porous nature of the pad also correlates with its compressible nature. The pad can be sized to fill the sample collection location and even have an uncompressed height that exceeds the height of the sample collection location such that, when the lid is closed, the pad is compressed. Alternatively, the pad can be a thin, single layer pad. The pores in the pad hold the sample fluid and also capture unwanted particulate contaminants so the pad can have a secondary function as a filter for larger particulate contaminants. The porous material may absorb the sample and holds the sample in the sample reception device until the lid is closed and the sample is forced from the porous material out of the sample collection location and into the decoupling zone.

In some embodiments, the pad of porous material may be passivated. Passivation of the pad of porous material may be intended to reduce binding of protein or other analytes.

In some embodiments, the pad of porous material may be removable. If the pad is removable, it can be placed in the mouth by the user and saturated with saliva. The size and absorbency of the pad will therefore dictate the size of the sample required. This removes the requirement on the user to estimate the correct sample size.

In some embodiments, the pad of porous material may be configured to provide visible indication when sample has been collected.

The controller may initiate the reaction timer when the pressure differential in the sample management module exceeds a predetermined value to bring the sample and reagent into contact.

This pressure differential may be a positive pressure difference provided by the closure of the device in which the sample management module is accommodated. This may be achieved manually by the user. Alternatively, it may be provided automatically when the device in which the sample management module is accommodated is introduced into an incubation and storage device or a reader through the use of a separate mechanism such as a plunger.

In some embodiments, the positive pressure may be provided by the operation of a pump located on the exterior of the device by the user. The pump may be a blister pump.

In some embodiments, the pressure differential may be a negative pressure difference which can be created by a pressure release vent or by opening a valve within the device. The vent or valve can be operable on demand to release stored pressure within the device, which can create a negative pressure difference sufficient to bring the sample and reagent into contact and actuate the controller to initiate a reaction timer. In some embodiments, once the vent or valve has been opened, it is capillary action alone that brings the sample and reagent into contact and initiates the assay.

Alternatively, the reaction timer may be initiated when a sliding gate is actuated enabling the fluid sample to flow over the reagent. In moving from a position that prevents the fluid sample from reaching the reagent to a position in which the fluid sample can come into contact with the reagent, the sliding gate can act as the controller and initiate the reaction timer.

The reagent may be a liquid reagent. The provision of the reagent in liquid form ensures that the reagent and the capture components from the sample fluid combine with ease and optimised mobility.

The sample management module may further comprise a wicking pad that accommodates the reagent. The provision of a wicking pad to accommodate the reagent creates a lateral flow response in the assay.

The sample management module may further comprise a permeable hydrophobic layer which becomes permeable when the pressure in the sample management module exceeds a predetermined value to bring the sample and reagent into contact.

The permeable hydrophobic layer prevents fluid from reaching the reagent and commencing the reaction until sufficient pressure is provided to the layer to cause it to allow the sample to flow through the layer and over the reagent.

The permeable hydrophobic layer may be an additional layer or the module may be configured such that the permeable hydrophobic layer is the flow resistor. The permeable hydrophobic layer may provide a limited amount of filtering and therefore may be provided instead of an additional filter.

The sample management module may further comprise a flow controller configured to manage the flow of sample over the reagent.

The flow controller can take any form that is effective in slowing the flow of the sample over the reagent. The bulk movement of the sample in the vicinity of the reagents needs to be slowed in order to enable the sample sufficiently so that the detection reagents can bind to the target components within the sample and then move to the capture components via diffusion. The flow controller may effectively halt the bulk fluid flow. Alternatively, the bulk fluid flow may be reduced to 1 mm/minute, 0.5 mm/minute, 0.25 mm/minute or even substantially 0.0 mm/minute, i.e. stationary, so that the diffusion of the components within the sample is significant.

The flow controller may be provided distally of the capture components. By placing the flow controller distally, or downstream, of the capture components, the flow of sample into the fluid pathway is unimpeded thereby enabling the sample to be quickly introduced into the cartridge. The flow controller then acts to slow the flow of the sample once it has reached the capture components.

The flow controller or flow restrictor can take any form that is effective in slowing the flow. The flow controller may be a capillary stop or a narrow or tortuous path. Alternatively, the flow controller may be provided by the geometry of the fluid pathway itself in the case where the fluid pathway is a well. The sidewall or sidewalls of the well provide the flow controller as they prevent the sample from flowing further and cause the sample to stop in the vicinity of the capture components that are applied to the base of the well or to the wall or walls near the base of the well.

The flow controller may comprise a capillary channel; a narrow or tortuous path; a capillary stop; a capillary stop with a vent or gas buffer or a flow resistor.

The flow controllers are selected to have no moving parts and therefore to control the flow as a direct result of their configuration. This is particularly appropriate for a point of care application where the device is in the hands of an unskilled operative. Under such circumstances, moving parts raise quality control issues and risks in relation to reliability that are difficult to mitigate. Flow controllers that lack moving parts are therefore selected where possible.

The sample management module described above may be incorporated in an assay chip, which may be an integrated chip. In this context, the term integrated chip is used to refer to a chip that includes all of the reagents required to complete all of the steps of the immunoassays that it is designed to process. This means that no further reagents need to be added to facilitate the processing of the immunoassay or the collection of the results. This also means that the chip does not require any washing steps, external pumping steps or other processing steps in order to complete the immunoassay and prepare the result for reading via the reader.

The reaction timer may be provided within the chip. The provision of the reaction timer within the sample management module ensures that the reaction can be timed directly without reliance of communication between the sample management module and any other aspect of the wider system in which the sample management module may be deployed.

The sample reception device may comprise a lid which acts as the controller in that it is configured to initiate the timer when the lid of the sample reception device is closed. The lid is configured so that, once closed, it cannot be reopened. Therefore the reaction timer can be initiated categorically on the closure of the lid because no further sample can be included after the commencement of the timer because, once the lid is closed, the user can no longer introduce sample into the device. This provides a considerable advantage over systems that are merely tamper-evident, because the lid physically prevents the user from accessing the device once the lid has been closed.

The sample management module may comprise an electrical circuit and completion of the circuit by closing the lid begins the timer. Alternatively, the electrical circuit may comprise a “normally closed” switch that is opened by the closure of the lid.

The chip may further comprise a unique identifier. The unique identifier may be a barcode, QR code or other visually addressable code that is capable of uniquely identifying an individual sample management module and associated chip.

The unique identifier may be editable. The unique identifier may be an RFID tag or other editable, readable tag. The editable nature of the unique identifier enables key information to be included on the tag such as the time of the commencement of the assay. This means that when the tag is read it provides the identity of the chip and also the time elapsed since the assay commenced. This information can then be conveyed to other parts of the system includes those parts where the results of the assay are read and where the data is subsequently processed and stored.

The data may be processed locally or centrally. The data, or metadata obtained through the processing of the data, may be communicated to the user as appropriate. For example, the user may be notified, via their smartphone, that the results from a sample align with the user’s normal level of whichever biomarkers have been monitored.

The data from the tag may be shared by near-field communication with the storage and incubation device and/or the reader.

The sample management module may further comprise a battery and the reaction timer may be a microprocessor.

The sample management module may comprise a transmitter configured to send the value of the timer to a reader device.

The sample may be a saliva sample. Providing a saliva sample is a simple, non-intrusive procedure. As a result, users are typically more willing to provide a saliva sample than, for example, a blood sample. Furthermore, this increases the frequency with which a user can be expected to provide a sample. By providing frequent samples, a personalised base line can be established for each user allowing a personal profile to be established and therefore feedback can be provided if level fall outside the expected levels for that individual. These can be a much tighter set of parameters than for the population as a whole.

The use of saliva as the sample fluid is also appropriate to a wider range of settings, for example the home or even the workplace. In some embodiments, all employees may be requested to provide a daily sample in order to look for pre-symptomatic flu. This can enable an employee to be sent home before symptoms develop, potentially reducing the number of colleagues infected by that individual and also potentially lessening the symptoms of the infected individual as a result of taking time off in advance of symptoms presenting.

The liquid sample may be any bodily fluid including, but not limited to blood, serum, plasma, semen or saliva. Different bodily fluids include varying levels of different biomarkers. Blood typically has relatively high concentrations of biomarkers of interest and therefore, in the laboratory setting, where biohazardous nature of processing and disposing of blood samples is not an issue there is a strong preference for using blood as the liquid sample because the biomarkers are present in relatively high concentrations and are therefore easily detectable.

However, in moving to the domestic or point of care setting with an unskilled operative and a relatively uncontrolled environment, blood processing becomes problematic. Furthermore, it is known that biomarkers tend to be present in much lower concentrations in saliva than they are in blood. There is therefore a considerable barrier to overcome in order to move from conventional blood testing to the use of saliva as the fluid sample. In order to detect biomarkers that are present in low concentrations, it is necessary to optimise the detectors to detect such low concentrations.

Providing a saliva sample is a simple, non-intrusive procedure. As a result, users are typically more willing to provide a saliva sample than, for example, a blood sample. Furthermore, this increases the frequency with which a user can be expected to provide a sample. By providing frequent samples, a personalised base line can be established for each user allowing a personal profile to be established and therefore feedback can be provided if level fall outside the expected levels for that individual. These can be a much tighter set of parameters than for the population as a whole.

The use of saliva as the sample fluid is also appropriate to a wider range of settings, for example the home or even the workplace. In some embodiments, all employees may be requested to provide a daily sample in order to look for pre-symptomatic flu. This can enable an employee to be sent home before symptoms develop, potentially reducing the number of colleagues infected by that individual and also potentially lessening the symptoms of the infected individual as a result of taking time off in advance of symptoms presenting.

The design of the assay cartridge or assay chip for use in this wider range of settings has to be appropriate to the setting. As a consumable item, for use by an end user, it has to be simple and intuitive to operate. It is designed to take a single sample from a user and then to store that sample and perform an immunoassay on that sample. As such, it entirely removes the risk of cross contamination as only one sample is provided in each assay cartridge or chip. The results of that assay can then be read, optically, by a reader into which the chip or cartridge is inserted. The chip or cartridge is provided with all of the reagents required to complete the immunoassay once the sample has been introduced. It can therefore be referred to as an integrated chip or integrated cartridge because no further reagents or wash fluid are required and then sample moves through the chip or cartridge without the need for external pumping.

The provision of a large number of samples, potentially in quite a small time window results in a need to store and sort the chips. In the above mentioned example, all employees may be requested to provide a sample on arrival at the workplace and therefore a large number of samples may be provided at a similar time.

Even though the reading part of the procedure is a relatively small amount of time, there remains a need to smooth the flow of chips in that an individual may have a number of chips in which the assays have been completed and may not wish to remain by the system in order to feed in each chip for reading sequentially. The user therefore needs to be able to present a number of chips all but simultaneously and the system needs to be able to accommodate these immediately and subsequently address the timing and reading of the assays.

The present invention will now be described, by way of example only, with reference to the accompanying figures in which:

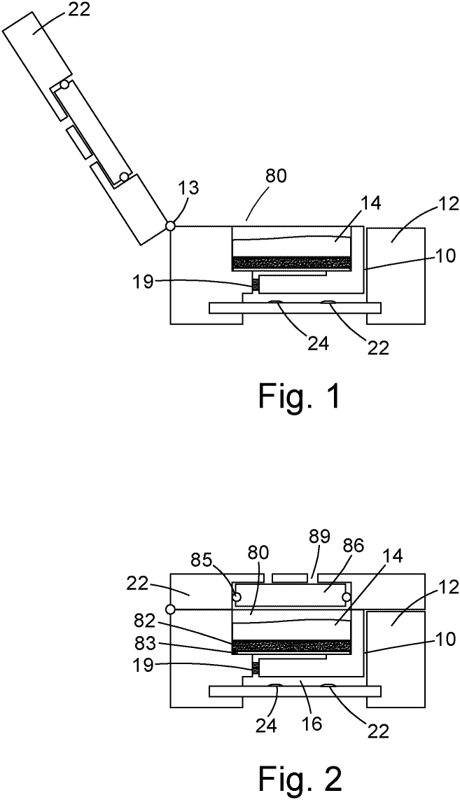

FIG. 1 shows schematically part of an assay chip during provision of a saliva sample;

FIG. 2 shows the assay chip in a closed position;

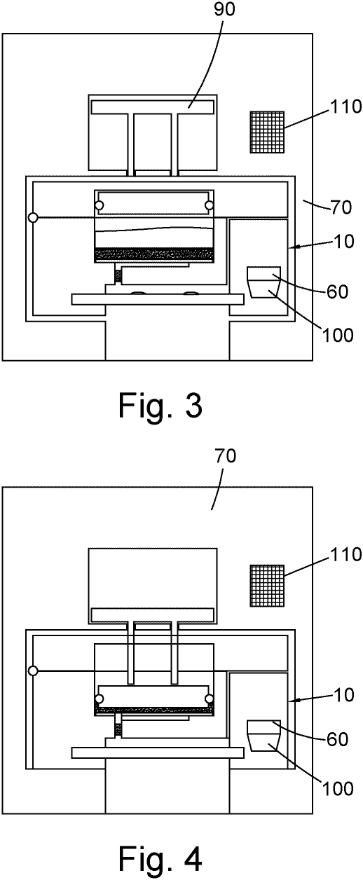

FIG. 3 shows the assay chip inserted in a reader;

FIG. 4 shows the assay chip actuated and the assay initiated;

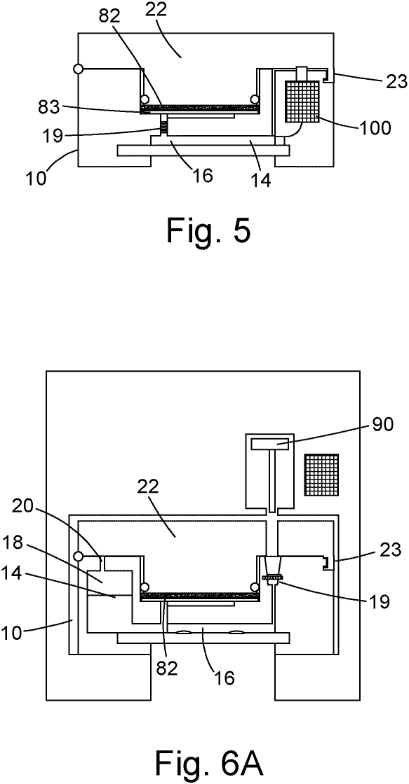

FIG. 5 shows an alternative assay chip;

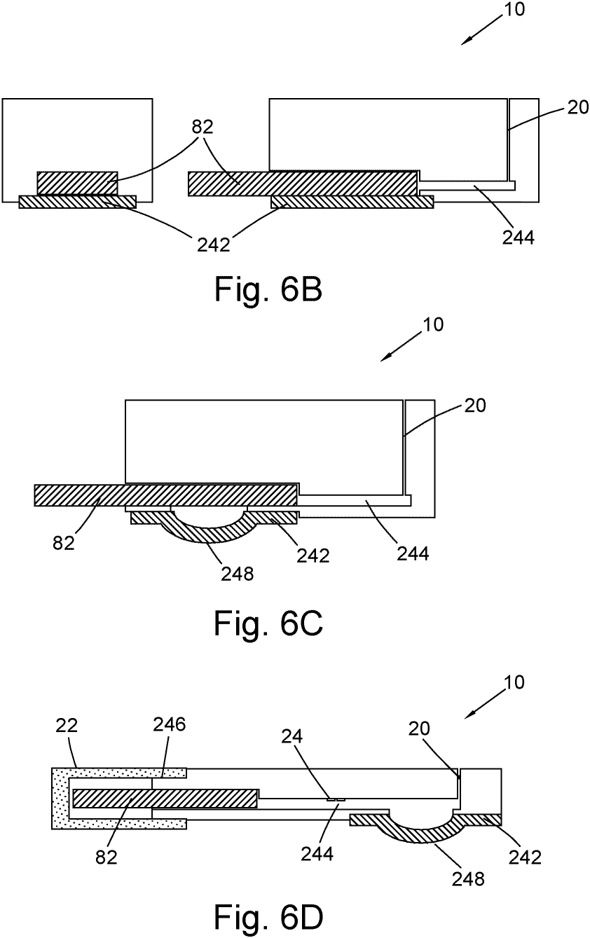

FIG. 6A shows a further alternative assay chip;

FIG. 6B shows a further alternative assay chip;

FIG. 6C shows a further alternative assay chip;

FIG. 6D shows a further alternative assay chip;

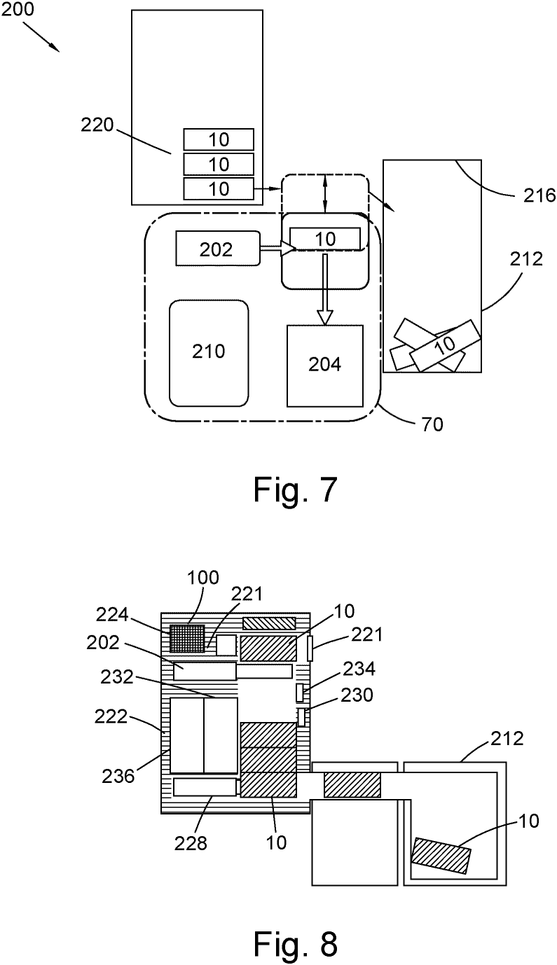

FIG. 7 shows a system for chip management;

FIG. 8 shows an example of a stacker that may form part of the chip management system of FIG. 7;

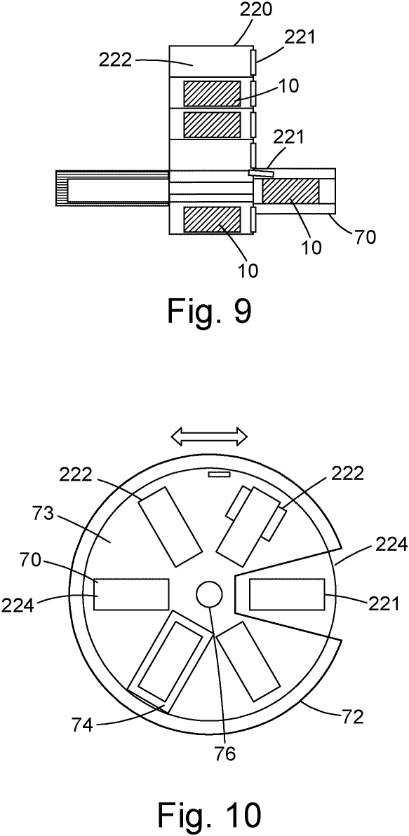

FIG. 9; shows an alternative example of a stacker that may form part of the chip management system of FIG. 7; and

FIG. 10 shows an alternative system for chip management.

FIG. 1 shows an assay chip 10, including a sample reception device 12 for receiving a liquid sample 14. The sample reception device 12 includes an opening 80 into which the liquid sample 14 is introduced. The sample reception device 12 also includes a lid 22 configured to cover the opening 80 once a liquid sample 14 has been provided. The lid 22 prevents the liquid sample 14 from exiting the chip 10. The lid 22 is attached to the assay chip 10 by a hinge 13. A hinged configuration is advantageous in that the lid 22 cannot be separated from the assay chip 10 and lost by the user. The hinge can be rigid, or can be non-rigid. In this context, the term hinge is understood to mean any configuration that maintains contact between the lid 22 and the assay chip 10 and provides relative movement between these parts so that the lid 22 can be placed onto the assay chip 10 by the user. The provision of a hinge of some sort ensures that the lid 22 cannot be separated from the assay chip 10 so the lid cannot be dropped or swapped. In an example not shown in the accompanying drawings, a flexible plastic portion may be used to attach the lid 22 onto the assay chip 10. This requires the user to have a given level of dexterity to bring the lid 22 into position on the assay chip 10. In another example not shown in the accompanying drawings, the hinge is a living hinge and the lid 22, the hinge, and the assay chip 10 are all the same material.

The sample reception device 12 is provided with a flow pathway 16 that links the opening 80 to the location of the detection reagents 24 and capture components 22. The fluid pathway 16 is provided with a flow controller or flow restrictor 19 that prevents the liquid sample 14 from moving along the fluid pathway 16. The flow controller 19 may be a hydrophobic pathway wall or a hydrophobic filter in the fluid pathway 16 or a widening of the cross section of the fluid pathway 16 such as to produce a capillary stop. The burst pressure of the flow controller 19, i.e. the pressure required to push the liquid past the flow controller, is determined by the contact angle and geometry as is known to experts in the field.

In the illustrated embodiment, in addition to the flow restrictor 19 there is a sponge 82 and or a filter 83 provided in the opening 80 where the liquid sample 14 is collected. In some embodiments, the functionality of these three items may be provided by one or two layers, for example a PTFE layer which may provide some filtering as well as restricting the flow of the sample. In embodiments such as that shown in FIG. 1 where it is provided as a separate item, the sponge 82 helps to locate the sample 14 and also provides a coarse grade filter to remove any unwanted particulate matter from the liquid sample 14. The filter 83 is downstream of the sponge 82 and filters out finer unwanted particulates from the sample.

As shown in FIG. 2, the lid 22 is moved to a closed position by the user. This ensures that the liquid sample 14 cannot leave the assay chip 10. The lid 22 does not necessarily provide an air tight seal, but it does provide sufficient barrier to exit that, in combination with surface tension, the lid 22 prevents at least majority of the liquid sample 14 from leaking out of the assay chip 10.

Located within the lid 22 is a plunger 86 which is held in place in a recess in the lid 22 by an O-ring 85. The plunger 86 is accessible via one or more through holes 89 adjacent to the plunger 86. There may be a single, annular through hole 89 or there may be a plurality of individual through holes 89 provided.

FIG. 3 shows the assay chip 10 inserted into a reader 70. The reader 70 is provided with a key 90 which is configured to match the through holes 89. Once the assay chip 10 has been inserted into the reader 70, the assay is commenced by the deployment of the key 90 initiating the plunger 86 via the through hole or through holes 89.

A controller 100 is provided to initiate a reaction timer 110 when the fluid sample 14 and the reagent 24 are brought into contact. The reaction timer 110 is provided in the reader 70 as the reader 70 will monitor the timing of the assay from the initiation of the assay, through the timing of the incubation phase and then identifying the correct time to take the reading of the results.

The controller 100 is provided as part of the sample management module that forms part of the assay chip 10. The controller 100 provides feedback to the reader that the key has effectively actuated the plunger to commence the assay and therefore the reaction timer, located on the reader 70 should be started.

The controller 100 also includes a unique identifier 60, the content of which can also be communicated to the reader 70. The unique identifier 60 may be passive, such as a QR code or barcode. If the unique identifier 60 is passive, then it can only be read and not written to. It is unique and has, as its primary purpose, to identify the specific assay chip on which it is provided. The identity may be made up of multiple pieces of information including the biomarkers provided as detection reagents and capture components and also the batch from which it is drawn. This information may be common to a number of chips. Within each batch, each chip then has a unique identifier 60 so that the results can be allocated to a specific user. The presence of a unique identifier 60 also provides an element of quality control because each assay chip can be identified and therefore it is possible to check whether a particular assay chip 10 has been tampered with or used incorrectly in any way.

In some embodiments, the unique identifier 60 is active and can be updated to include information about the progress of the assay. In embodiments in which the unique identifier 60 is editable, it will include some form of memory enabling a data storage facility. The data storage may be in any suitable form of memory, either volatile or non-volatile, including but not limited to RAM, SRAM, ROM, EEPROM or Flash memory. For example, the unique identifier may be an RFID tag.

FIG. 5 shows schematically a further embodiment of the chip 10 shown in FIGS. 1 and 2. There is a considerable commonality of components between the embodiments. The key difference between the embodiments is the functionality of the controller 100 and the location of the reaction timer 110. In this embodiment, the lid 22 is provided with a one way latch or clip 23 so that, once closed, it cannot be reopened without the application of disproportionate force and the plunger 86 is provided in a recess in the lid 22. The plunger 86 and sponge 82 are sized such that, when the lid 22 is closed the plunger 86 compresses the sponge 82 providing sufficient pressure to overcome the flow restrictor 19 and introduce the fluid sample 14 into the fluid pathway 16 in which the detection reagents 24 and capture components 22 are provided. In this way, the closure of the lid 22 by the user, commences the assay.

The reaction timer 110 is included on the assay chip 10 and the controller 100 includes a circuit that is completed by the closure of the lid which thereby commences the reaction timer 110. The data relating to the timing of the assay is stored in the controller 100 or on the unique identifier, if it is editable, for communication to the reader 70. In this embodiment, the assay chip 10 does not need to be introduced to the reader 70 until the assay has been completed.

The fluid pathway 16 can be provided with a sensor (not shown) that identifies when the sample fluid reaches the detection reagents 24. The sensor can be capacitive or conductive. In some embodiments, the closure of the lid and the commencement of the assay occur effectively at the same time. This is the case where the assay time is quite long, for example 8 hours or 12 hours and therefore the seconds or minutes that it takes for the liquid sample to come into contact with the detection reagents and thereby to commence the assay is a negligibly small percentage of the assay time. In some embodiments, although the time taken for the liquid sample to reach the detection reagents and therefore commence the assay is a statistically significant proportion of the assay time, it is a known quantity which is reasonably repeatable between assays. In those embodiments where neither of the above applies, then the provision of an additional sensor in the fluid pathway provides additional certainty as to the exact timing of the commencement of the assay.

FIGS. 6A to D show further embodiments of the assay chip 10 shown in FIGS. 1 and 2. Although there is a high degree of commonality of features between the embodiments of FIGS. 1, 2 and 6, the control of the initiation of the assay is different in the embodiments of FIGS. 6. In FIG. 6A, the flow controller 19 is a burstable layer such as a plastic film that, once burst allows a capillary channel to draw passively the fluid sample 14 past the detection reagents 24. The flow controller 19 is actuated by a key 90 which bursts the layer either through physical contact, such as the key 90 having a sharp protrusion or through the application of radiation, for example, laser light.

This configuration allows the lid 22 to be closed and therefore the sample encapsulated from further contamination, but the assay not commenced. The chip 10 can therefore be stored in this configuration with the liquid sample 14 contained within the assay chip 10 for the assay to be run at a later time. The actuation of the assay would therefore occur only when the key 90 was activated, either on introduction of the assay chip 10 into a storage and incubation device or reader.

Whilst the liquid sample 14 is held in the assay chip 10 prior to the bursting open of the flow controller 19 to enable the commencement of the assay, the fluid sample 14 is retained in a reservoir 18 and the pressure within the reservoir 18 is managed by the provision of a vent 20.

FIG. 6B shows a further embodiment of the assay chip 10 shown in FIGS. 1, 2 and 6A. The chip 10 of FIG. 6B has many common features with the chips shown in FIGS. 1, 2 and 6A. In the embodiment of FIG. 6B, a sponge 82 is inserted by the user into the assay chip 10 after wetting with a liquid sample 14. Alternatively, the sponge 82 may be pre-assembled in the assay chip 10 leaving one end of the sponge 82 exposed so that the user can wet the sponge 82 with a liquid sample 14 without removing the sponge 82 from the assay chip 10. The assay is initiated by compression of the sponge 82, which advances the liquid sample 14 through the sponge 82, past the detection reagents 24 and into an analysis chamber 244. To enable the compression of the sponge 82, the assay chip 10 comprises a flexible wall 242. The sponge 82 is compressed by applying a pushing, sliding or rolling action to the flexible wall 242, either manually or by an automated process. A roller, blade or piston can be used. Alternatively or additionally, the flexible wall 242 can be manipulated manually with a finger. Alternatively or additionally, the assay chip 10 may have an opening (not shown) enabling compression of the sponge 82.

The flexible wall 242 may be shaped to form a blister or bellow 248 which is filled with air or gas, as shown in FIG. 6C and FIG. 6D. FIG. 6C shows an example in which the blister 248 is pushed manually or by an automated process to apply pressure to the sponge 82, and move the liquid sample 14 past detection reagents 24 and into the analysis chamber 244. The pressure within the chip 10 is managed by the provision of a vent 20.

Alternatively, the blister 248 in the flexible wall 242 may contain a burstable liquid pouch (not shown) containing either a buffer solution or the detection reagents 24. The burstable liquid pouch within the blister 248 can be configured to burst upon application of pressure, either manually or by an automated process. Alternatively the blister 248 and burstable liquid pouch can be punctured by a sharp object (not shown). The act of bursting the burstable liquid pouch releases either the buffer solution or detection reagents 24 and is the actuation event which initiates the reaction timer.

FIG. 6D shows a blister 248 in the flexible wall 242, which functions as a vacuum pump. The assay chip 10 is substantially cylindrical and includes a lid 22 which covers the sponge 82 once a liquid sample 14 has been provided. The lid 22 is provided with a one way latch or clip (not shown) so that, once closed, it cannot be reopened without the application of disproportionate force. When closed, the lid 22 can overlap with the sides of the assay chip 246 to provide a sufficient barrier to exit and prevent at least the majority of the liquid sample 14 from leaking out of the assay chip 10. The lid 22 allows the device to be closed and therefore the sample encapsulated from further contamination, but the assay not commenced. The actuation event occurs as the blister 248, which is filled with air or gas is compressed and forms a negative pressure differential which draws the sample and detection reagent 24 into contact. The sponge 82 is wetted with the liquid sample 14 prior to compression of the blister 248, which blocks the displaced air from escaping via the pores of the sponge 82. The air or gas is forced through a vent 20. The vent 20 is a one way valve. The elastic properties of the flexible wall 242 create a negative pressure difference when the blister 248 is released. This pressure differential draws the liquid sample 14 from the sponge 82 and into the analysis chamber 244 where the detection reagents 24 are placed. This actuation of the blister 248 can be manually achieved by a user or it may be automatically initiated by an incubation and storage device at any time. FIG. 7 shows a system 200 for chip management. The system 200 includes a reader 70 which comprises an illumination device such as a laser 202 and a detection device such as a camera 204. The laser 202 and the camera 204 are both directed to a berth or location at which an assay chip 10 is provided to be read. There is also a controller 210 that communicates with a storage and incubation device 220. The system 200 also includes a waste chip bin 212.

The storage and incubation device 220 includes a plurality of berths 222 each sized to accommodate an assay chip 10; a scanner 224 configured to read the unique identifier 60 of each assay chip 10; a clock timer 226 configured to monitor the timing of the assay within each assay chip 10; and a completion module 228 to manage each assay chip 10 when the assay is complete.

The scanner 224 is provided for reader data from the unique identifier 60. The information includes, at a minimum, the identity of the assay chip and an indication as to the timing regimen in use. For example, the data provided with the unique identifier may identify the batch from which the assay chip has been drawn, together with a unique identifier for the single assay chip. The data can then include the fact that the timing cannot be carried out on the assay chip because the assay chip in question does not have a reaction timer. This information can then be processed within the storage and incubation device 200 to ensure that the clock timer 226 is timing the entire assay as there is no provision on the assay chip 10 to time the assay locally.

The clock timer 226 times the incubation of the assay on each assay chip 10 that is introduced into the storage and incubation device 220. Depending on the initiation of the assay, the clock timer 226 may time the entire assay from start to finish or it may align itself with a reaction timer provided on the assay chip 10. In the later circumstance, it will time only the later part of the assay, commencing its monitoring at the point where the assay chip is introduced into the storage and incubation device 220.

A processor 225 is provided to write data to the unique identifier tag 60 where appropriate. Where the unique identifier is an editable tag with a writable memory, the processor 225 provides information about the incubation and storage of the assay chip to the unique identifier 60. This can include, but is not limited to timing information and identity information about the storage and incubation device. This information may then be accessed by the reader 70 directly from the assay chip 10 when the reader 70 obtains the results of the assay. The data stored may be preprocessed by the processor 225 within the storage and incubation device 220 in order to minimise the number of write operations required and also to reduce or eliminate the processing required on the assay chip 10 itself which may have very limited computational or battery capability. Therefore, where possible, the processor 225 within the storage and incubation device 220 processes the data such that only a summary, or meta-data, is written to the assay chip 10.

FIG. 8 shows an example of the storage and incubation device 220. In this embodiment the berths 222 are located in a linear stack that is held by gravity so that each assay chip 10 falls down by gravity onto the completion module 228. The berths 222 comprise one or more features, such as slots or grooves to match the shape or configuration of the assay chip and therefore to aid alignment. Alternatively or additionally, the movement of the chips 10 within the storage and incubation device 220 may be managed by a spring force against a latch 223 that can release a chip 10 to be transferred to the reader 70. The completion module 228 of this embodiment of the storage and incubation device 220 is a linear actuator or conveyor which is configured to make a linear translation of the chip 10 so that it exits the storage and incubation device 220 and moves into the reader 70.

The storage and incubation device 220 includes a loading slot 221. The loading slot 221 guides the assay chip 10 into position on one of the berths 222 provided within the storage and incubation device 220. Each of the berths is sized to accommodate an assay chip 10. The loading slot 221 is sized to compress the assay chip 10 thereby forcing the latch 23 to close the lid 22 of the assay chip 10 and the commencement of the assay. This may be achieved by the loading slot 221 being only just sufficiently large to accommodate the assay chip 10 and thereby to provide pressure around the circumference of the assay chip 10. Alternatively, the loading slot can include a projection at a key point on the circumference of the loading slot such that pressure is applied to a key part of the assay chip, adjacent to the latch 23 on the lid 22 of the assay chip 10.

The storage and incubation device 220 also includes a temperature sensor 230 and temperature controller 232. The rate at which assays progress is dependent on the temperature at which they are incubated. Some assays have a wide operating envelope with regard to temperature. In these circumstances it may be sufficient to monitor the temperature. Information about the temperature may be logged in the memory 62 associated with the controller 100 of the assay chip 10. This data can be used to identify when the assay will be complete. For example, if the assay proceeds more rapidly at an elevated temperature, then the assay will have progressed sufficiently to take a reading at an earlier time than would be the case at a lower temperature.

If the assay has a more narrow operating envelope, then the temperature controller 232 is used to modulate the temperature to ensure that the temperature remains within the required operating envelope of the assay.

The temperature controller 232 can also be used in circumstances where the operating envelope is wider, but the unique identifier 60 is not editable. In these embodiments, the temperature data cannot easily be stored on the assay chip 10 and therefore the rate at which the assay progresses has to be more actively controlled to ensure that it progresses at a predetermined rate by raising or lowering the temperature to a predetermined level.

A humidity sensor 234 and humidity controller 236 are provided. As with temperature sensing and control as outlined above, some assays are fairly resilient as to the humidity and a monitoring approach may be sufficient. In circumstances where the assay is more sensitive to the humidity, the humidity controller can be activated to maintain the humidity within a tighter acceptable operating window.

The completion module 228 may transfer the assay chips 10 to the waste chip bin 212. The waste chip bin 212 is a collection location for used chips. Depending on the chip contents, the chips may be removed from this location and disposed of in household waste by the user. However, the chips may be recycled and therefore the user may be expected to send them back to the service provider where they may be recommissioned or recycled in whole or in part. In order to alert the user to the need to empty the waste chip bin 212, there is a sensor 214 and an alarm 216 to communicate to the user that the waste chip bin needs to be emptied.

The completion module 228 includes a moving plate 229 that acts as a transferring element, linear actuator or conveyor configured to move the assay chip 10 from the storage and incubation device 220 and into the reader 70.

Movement of the assay chip 10 from the reader 70 into the waste chip bin 212 can either occur on a continuation of the moving plate 229 or by pressure in that the introduction of a subsequent assay chip 10 into the reader 70 displaces the incumbent assay chip 10 and ejects it into the waste chip bin 212.

FIG. 9 shows an alternative embodiment of the storage and incubation device 220 that is provided with a plurality of loading slots 221. Each loading slot 221 is provided adjacent to a corresponding berth 222 onto which an assay chip 10 can be delivered. The entire storage and incubation device 220 is then configured to move relative to the reader 70 so that the outlet of the reader 70 can be positioned adjacent to any one of the loading slots 221. The storage and incubation device 220 is provided with a mechanism that ejects an assay chip 10 from one of the berths 222 through the loading slot and directly into the reader 70. In this configuration the loading slot 221 is also effectively an unloading slot as the assay chip 10 both arrives and leaves the storage and incubation device 220 through the same slot 221.

FIG. 10 shows an alternative configuration in which the storage and incubation device 220 and the reader 70 are accommodated within a single housing 72. The housing 72 is substantially cylindrical and the berths 222 are configured on a planar circular surface 73 that can rotate around the axis 76 of the cylinder. The rotation of the berths 222 is indexed so that the berths 222 can stop in one of a predetermined number of positions around the circle. Each predetermined location at which a berth can stop has a defined function. One of the locations includes the loading slot 221 which may be an indentation in the housing 72 configured to expose the berth 222 indexed to the loading location. One of the locations includes the reader 70 so that the results of the assay on the assay chip 10 can be read when the assay chip 10 is indexed to the reading position. One of the locations includes a waste shoot 74 from which assay chips 10 may be sent to the waste chip bin, not shown in this embodiment as it is located beneath the berths 222. All of the other locations are used for incubation.

Although a total of six locations are shown in FIG. 10, the device 220 can be configured with any number of locations as appropriate. There must be at least three locations: loading, reading and ejection to waste. The scanner 224 can be positioned to interrogate the assay chip 10 in the loading or reading location. In most examples there will be at least four locations so that there is at least one dedicated incubation location. There may be one, two, three, four, five, ten or up to twenty incubation locations, but accommodating many more makes the design cumbersome as the required diameter increases to accommodate all of the incubation locations.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A sample management module for receiving a fluid sample for an immunoassay, the module comprising:
a sample reception device for receiving a sample;
a detection reagent;
a controller configured to initiate a reaction timer on the basis of an actuation event when the sample and detection reagent are brought into contact.

2. The sample management module according to claim 1, wherein the controller initiates the reaction timer when the pressure differential in the sample management module exceeds a predetermined value to bring the sample and reagent into contact.

3. The sample management module according to claim 1 or claim 2, wherein the reagent is a liquid reagent.

4. The sample management module according to claim 1 or claim 2, further comprising a wicking pad that accommodates the reagent.

5. The sample management module according to claim 2, further comprising a permeable hydrophobic layer and wherein the permeable hydrophobic layer becomes permeable when the pressure in the sample management module exceeds a predetermined value to bring the sample and reagent into contact.

6. The sample management module according to any one of claims 1 to 5, further comprising a flow controller configured to manage the flow of sample over the reagent.

7. The sample management module according to claim 6, wherein the flow controller comprises a capillary channel; a narrow or tortuous path; a capillary stop; a capillary stop with a vent or gas buffer or a flow resistor.

8. An assay chip comprising a sample management module according to any one of claims 1 to 7.

9. The assay chip according to claim 8, wherein the chip is an integrated chip.

10. The assay chip according to claim 8 or claim 9, wherein the reaction timer is provided within the chip.

11. The assay chip according to any one of claims 8 to 10, wherein the sample reception device comprises a lid which acts as the controller in that it is configured to initiate the timer when the lid of the sample reception device is closed.

12. The assay chip according to claim 11, wherein the sample management module comprises an electrical circuit and completion of the circuit by closing the lid begins the timer.

13. The assay chip according to any one of claims 7 to 12, further comprising a unique identifier.

14. The assay chip according to claim 13, wherein the unique identifier is editable.

15. The assay chip according to any one of claims 7 to 14, wherein the sample management module further comprises a battery and the reaction timer is a microprocessor.

16. The assay chip according to any one of claims 7 to 15, wherein the sample management module comprises a transmitter configured to send the value of the timer to a reader device.

17. The assay chip according to any one of claims 7 to 16, wherein the sample is a saliva sample.
